(19) United States
(12) Patent Application Publication
(10) Pub. No.: US 2017/0312309 A1
(43) Pub. Date: Nov. 2, 2017

(54) INTRADIALYTIC ADMINISTRATION OF SODIUM THIOSULFATE

(71) Applicant: Anupkumar Shetty, Irving, TX (US)

(72) Inventor: Anupkumar Shetty, Irving, TX (US)

(21) Appl. No.: 15/651,478

(22) Filed: Jul. 17, 2017

Related U.S. Application Data
(63) Continuation of application No. 14/884,084, filed on Oct. 15, 2015, now abandoned.
Application No. 14/884,084 is a continuation of application No. 12/157,140, filed on Jun. 9, 2008, now abandoned.

(51) Int. Cl. A61K 33/04; A61M 1/16; A61K 49/06; A61M 1/28; A61K 33/00

(52) U.S. Cl. CPC A61K 33/04; A61M 1/287; A61K 33/00; A61K 49/06; A61M 1/1654

(57) ABSTRACT

The invention provides a source of sodium thiosulfate via the dialysate used to cleanse the bool of toxic and metabolic waste in the patients undergoing hemodialysis, peritoneal dialysis, or gastro-intestinal dialysis for treatment of end-stage or near end-stage chronic renal disease. In the method of the invention, dialysis solution components contain therapeutic amounts of sodium thiosulfate, which when fully reconstituted for use as a single solution, deliver 20-130 mg/dl of dialysate.

### BACKGROUND OF THE INVENTION

Patients with chronic kidney disease (CKD) progress through different series of stages before they need dialysis or kidney transplantation. Kidney disease outcomes quality initiative (KDOQI) from the National Kidney Foundation has classified kidney disease into stages 1 to 5 depending upon the degree of renal function (K/DOQI clinical practice guidelines for chronic kidney disease: evaluation, classification, and stratification. Am J Kidney Dis. February; 39(2 Suppl 1):S1-266). Dialysis is required to maintain homeostasis in patients with stage 5 chronic kidney disease. Patients with chronic kidney disease stage 5 on dialysis are stated to be in end stage renal disease (hereafter called ESRD). Dialysis is defined as the movement of solute and water through a semipermeable membrane which separates the patient's blood from the dialysate solution. The semipermeable membrane can either be the peritoneal membrane in peritoneal dialysis patients or an artificial dialyzer membrane in hemodialysis patients or the gastrointestinal mucous membrane in gastrointestinal dialysis.

The data from United States Renal Data System suggests that the annual mortality of patients on dialysis is more than 20% and has remained so for the last two decades with minimal improvement (data available at www.usrds.org). This is higher than the mortality rate of many common cancers such as breast cancer, colon cancer and prostate cancer.

Vascular and extraosseous calcification is a common event seen in patients with chronic kidney disease and is responsible for most of the morbidity is patients with CKD. Cardiovascular events are responsible for over 50% of the mortality in hemodialysis population. Coronary calcification is almost a universal observation in majority of the patients on hemodialysis above the age of 20 years (Goodman W G, et al., N Eng J Med 2000; 342: 1478-1483) and it tends to progress with vintage on dialysis (Goodman W G, et al., N Eng J Med 2000; 342: 1478-1483). End Stage Renal Disease (ESRD) is associated with a very high prevalence of coronary artery disease responsible for high coronary morbidity and mortality. In addition to the high prevalence of neointimal calcification of atherosclerosis, which by itself is associated with high prevalence of coronary artery disease in general population, patients with ESRD also have medial calcification, also called Monckeberg's atherosclerosis. Medial calcification is believed to be one of the causes of narrowing or occlusion of these arteries. High coronary artery score (hereafter called CAC score the risk of myocardial infarction in patients with ESRD [Raggi, et al., J. Am. Coll. Cardiol., 39: 695 (2002)] and this same study has also shown high incidence of aortic or mitral valve calcification in ESRD and its direct correlation to vintage on dialysis.

Vascular calcification is also responsible for devastatingly painful skin ulcers, which increase the suffering, morbidity and mortality of the affected patients. This can also contribute to limb loss in many of these patients. It is likely that arteriolar calcification contributes to ischemic events elsewhere in the body, which can result in devastating and sometimes fatal events such as stroke (Vlierenthart R, et al., Stroke. 2002;33:462) and ischemic colitis.

In addition, pulmonary parenchymal and vascular calcification contributes to restrictive lung disease and pulmonary hypertension both of which are common in patients with chronic kidney disease contributing to their morbidity and mortality.

Vascular and ectopic tissue calcification probably has a role in painful neuropathy, neuropathic ulcers, muscle weakness, musculoskeletal pain, vascular access problems and possibly fractures. Theoretically, calcification in the tissues and blood vessels can have a role in majority of the non-infective problems commonly encountered in patients on dialysis.

An effective way of treating and/or preventing vascular and ectopic calcification in patients with chronic kidney disease of different degrees has a potential to improve the morbidity and mortality of this group of patients. It is also known that those with coronary calcification have higher prevalence of coronary ischemic events (Vlierenthart R, et al., European Heart Journal 2002 23(20):1596-1603) and coronary calcification precedes coronary events.

Vascular calcification is a complicated process associated with transformation of vascular smooth muscle cells into osteoblast-like cells, which lay down a bone matrix of type I collagen and noncollagenous proteins. This framework acts as a nidus for mineralization, which results in calcification of the vessel and subsequent ischemia (Moe S M, et al., Pediatr Nephrol 18:969-975, 2003). The mineral deposited in the blood vessels is believed to have the same physicochemical properties of hydroxyapatite, the mineral compound of bone. While the exact cause of vascular calcification in uremic patients is unclear, there are multitudes of factors, which can be blamed. Some of these factors include hyperparathyroidism, oxidant injury, high amount of circulating calcium in patients with low PTH levels, positive calcium balance during hemodialysis, alkalemia increasing the synthesis of amorphous calcium phosphate and its further conversion into apatite, decrease in the concentration of pyrophosphate into orthophosphate by increased alkaline phosphatase activity (pyrophosphate is an inhibitor of conversion of amorphous calcium phosphate into apatite), decrease in y carboxylation of matrix Gla protein and others. A review of case-control series in adult patients demonstrated that hyperphosphatemia but not hypercalcemia or hyperparathyroidism was a risk factor in the development of calcemic uremic arteriolopathy (CUA) (Moe S M, et al., Pediatr Nephrol., 19:969 (2003). Other risk factors for CUA include female gender, white race, hypoalbuminemia, and warfarin use.

Calcification consists of noncrystalline (or amorphous) calcium phosphate, whitlockite ([Mg,Ca]₃[PO₄]₂), apatite (Ca₁₀[PO₄]₆[OH]₂) and hydroxyapatite ([Mg,Ca]₁₀[PO₄,CO₃]₆[OH]₂). Apatite is the predominant crystalline form in blood vessels. While formation of amorphous calcium phosphate is reversible, the formation of whitlockite, apatite and hydroxyapatite is irreversible under physiologic conditions. Alkalemia at levels that occur after hemodialysis favors both conversion of Ca₂+ and HPO₄²− into amorphous calcium phosphate and formation of apatite. Pyrophosphate and y-carboxylated matrix Gla protein (MGP) are inhibitors of conversion of amorphous calcium phosphate into apatite. Magnesium inhibits the formation of apatite, but increases the formation of whitlockite. These mechanisms have been reviewed in a simplified manner in a recent commentary (O'Neill, Kidney Internat., 71: 282 (2007)). A recent study by Verberckmoes et al (Kidney Internat., 71: 298 (2007) showed that whitlockite was only present in calcium deposits of uremic vessels of rats treated with calcitriol and not in uremic rats not treated with calcitriol.

In the prevention and treatment of conditions involving calcification in renal patients, there are very few specific remedies because the pathological processes are not well understood. In general, calcification is slowed or abated by careful monitoring and adjustment of circulating phosphate and calcium, so that a proper ion balance is maintained. In some cases, increasing the number and duration of dialysis episodes is recommended.

There have been a few isolated reports of efficacy in controlling nephrolithiasis (Yatzidis, Clin. Nephrol., 23:63 (1985), renal tubular acidosis with nephrocalcinosis (Agroyannis, Scand. J. Urol Nephrol., 28: 107 (1994), and calciphylaxis (Brucculeri, et al., Sem. in Dialysis, 18: 431(2005) by administering sodium thiosulfate either orally or intravenously.

Sodium thiosulfate has a small molecular weight of 248 (Na₂S₂O₃) and in patients with normal renal function has a serum half-life of 15 min. Animal data, using normal and anuric mongrel dogs, demonstrated that sodium thiosulfate distributes rapidly throughout the extracellular space (Braverman, et al., Proc. Soc. Exp. Biol. Med. 70:273 (1982). During renal failure, its volume of distribution doubled and the metabolic clearance rate decreased drastically. In the normal animals, sodium thiosulfate had a half-life of 46.8 min and >98% was cleared renally. However, in anuric dogs the half-life was 239 min and sodium thiosulfate elimination was primarily through the biliary system. In six healthy humans, the average volume of distribution of sodium thiosulfate was found to be 12.2 L (167 ml/kg), whereas in edematous individuals, it was 18.2 L (240 ml/kg) (Cardozo R H, et al., J Clin Invest 31:280-290, 1952). Brucculeri et al. (Brucculeri M, et al., Semin Dial 18:431-434, 2005) measured serum sodium thiosulfate concentrations in a patient with ESRD 15 min after infusion, before hemodialysis (52 h after administration) and after a 4-h hemodialysis session. The recorded sodium thiosulfate levels were 110, 1.2, and 0 μg/ml, respectively, with a calculated half life of 478 minutes.

Pharmacokinetic data on sodium thiosulfate (hereafter called STS) during other forms of renal replacement therapy, including peritoneal dialysis, are lacking.

At this time there is no effective treatment for arteriolar calcification. In uremic patients with severe hyperparathyroidism and cutaneous arteriolar calcification with calciphylaxis, emergent parathyroidectomy is known to show prompt relief. However arteriolar calcification has also been reported in many patients with normal or low PTH levels as well. There is no satisfactory treatment for the arterial calcification in these patients. Although the exact role of calcium in the pathogenesis of Uremic Arteriolar Calcification is not clear, therapies for CUA have included parathyroidectomy, use of non-calcium-containing phosphate binders, avoidance of administration of vitamin D analogs, and use of low-calcium dialysate for patients who are on intermittent hemodialysis. Recent retrospective data showing correlation between vitamin D usage and lower mortality in dialysis patients has received lot of attention even though the protective effects of vitamin D have not been proven in prospective studies. Moreover there is data in uremic rats showing extra osseous calcification induced by the commonly prescribed analogues of vitamin D such as calcitriol, doxercalciferol and pericalcitol. There is a possibility of inducing more calcification in patients with ESRD with potential harm if these retrospective studies drive the vitamin D utilization up.

Since the annual mortality of dialysis patients has not improved much over the years and since extra osseous calcification is a common phenomenon in ESRD, treating and preventing extra osseous calcification is a potential way of improving the outcome of these patients.

### SUMMARY OF THE INVENTION

The patient base to which the invention applies is primarily ESRD patients who are placed on regular dialysis. This includes patients with some or no renal function who undergo intermittent dialysis to reduce wastes from the blood to safe levels. It is a fundamental object of the invention to provide protective and preventative therapy to patients not yet presenting with acute symptoms of calcification in its various clinical forms, particularly those with a high coronary calcification score, any confirmed calcification in any artery, and certain high risk individuals such as diabetics.

It is another object of the invention to prevent disease states that are associated or correlated with calcification such as coronary and cerebral ischemic events, restrictive lung disease, peripheral vascular occlusive disease, systolic or diastolic dysfunction, cardiac arrhythmia, pulmonary calcification, muscular calcification, and a host of other disease states known to those skilled in the art.

A still further object is to provide a treatment where the foregoing disease states have already commenced but have not yet progressed to acute injury. In any patient population there will be a range of severity of pathology, and it is within the scope of the present invention to make adjustments in therapy in response to the individual's condition.

In the present invention, sodium thiosulfate is made available to the patient undergoing regular dialysis by formulating conventional dialysates to contain it in a therapeutically effective amount. Most conveniently, sodium thiosulfate can be formulated into standard, conventional dialysate concentrates, so that the desired concentration is obtained when the dialysate is fully reconstituted in the final dialysate. Alternatively, sodium thiosulfate can be added as a percentage by weight of the dry or liquid dialysate concentrate, or admixed into the liquid dialysate fully reconstituted. The concentration of sodium thiosulfate in the 1× strength dialysate is in the range of about 20 mg/dl to about 130 mg/dl. The dialysate Bicarbonate component concentrate contains from about 2.3 percent dry compound to about 15.5 percent dry sodium thiosulfate. These compositional ranges are intended as guidelines, but those skilled in the art will understand when the clinical condition of individual patients require an adjustment of concentration.

The invention further provides a method of treating dialysis dependent patients suffering from end stage or near end stage renal disease where they have or may be susceptible to complications from calcification processes in the body, by dialyzing them with the sodium thiofulfate-containing dialysate noted above. The method of the invention encompasses all the forms of dialysis including hemodialysis, peritoneal dialysis, and gastrointestinal dialysis. In peritoneal dialysis, use of sodium thiosulfate has a cytoprotective effect on the mesenteric membranes, the result of its chelating, antioxidant, and reducing properties.

These properties also have particular efficacy in patients suffering from nephrogenic fibrosing dermopathy and undergoing magnetic resonance imaging, to hasten removal of gadolinium from the body and hasten healing.

Calcification processes treatable or preventable by the present method, and associated with particular disease states include coronary ischemic events, cerebro vascular ischemic events, peripheral vascular occlusive disease, mesenteric ischemia, restrictive lung disease or hypertension, skin ulcers, decubitous ulcers, and other organ vascular insufficiency, myocardial and pericardial calcification, endocardial and cardiac valvular calcification, muscular and skeletal calcification, and calcification of perineurium, endoeurium, the vasa nervorum, distal pre-capillary arterioles, and soft tissue. This list is intended to be somewhat representative, but not all inclusive, of calcification-related conditions treatable and preventable by use of the invention.

In another embodiment of the inventive method, dialysis employing the sodium thiosulfate-containing dialysate can be performed on a patient undergoing magnetic resonance imaging using gadolinium as an imaging agent, in order to hasten removal of gadolinium from the body.

The advantages of the present product and method include ease of administration at minimal cost. The drug is provided simultaneously with another procedure for which the patient has no other option than death (or transplant). Given the half life of the drug, the dialysis sessions provide sufficient time for it to contact the target body tissues before dissipation and/or elimination. Third, the drug is administered by a health care professional thus obviating the chance of skipped doses, or overdosing. Finally, the present method obviates the need for any separate procedure different from what the patient normally experiences.

### DETAILED DESCRIPTION OF THE PREFERRED EMBODIMENTS

In accordance with the present invention, a method is provided for the administration of sodium thiosulfate to dialysis patients during the dialysis treatment. This administration can be utilized for patients on hemodialysis (acute or maintenance) or peritoneal dialysis (acute or maintenance) or gastrointestinal dialysis (acute or maintenance).

More specifically, dialysis patients are those patients undergoing hemodialysis or peritoneal dialysis or gastrointestinal dialysis for advanced CKD. Long-term dialysis therapy for treatment of end stage renal failure is referred to as maintenance dialysis. Almost all of the patients on maintenance hemodialysis above the age of 20 yrs have been shown to have a high coronary calcification score and the annual mortality of patients with ESRD is over 20%. Over fifty percent of the deaths in ESRD are from cardiac causes. Over 90% of the patients with end stage renal disease in the United States generally receive hemodialysis three times per week. A little less than 10% of these patients receive peritoneal dialysis in the US. The relative utilization of different modalities of dialysis variable in the different countries around the world.

A specific example of a conventional hemodialysis system is the Fresenius system. In the Fresenius system using NATURALYTE® 9000Rx-12 as the dry bicarbonate powder mixture (Catalog No. 08-9100-2) and GRANUFLOW® NATURALYTE® dry acid concentrate (Cat. No OFD 1067-3B), the ratio of acid:bicarbonate:purified water is 1:1.83:34. Therefore, one part of the concentrated bicarbonate solution is mixed with 19.126 parts of the other (acid+water), to make the final dialysate. In order to make the bicarbonate concentrate, purified water that meets or exceeds the current AAMI/ANSI hemodialysis water quality standards (hereafter called ‘purified water) is used. Fresenius supplies sodium bicarbonate powder packaged in bags and the contents of each bag containing 6264 grams of sodium bicarbonate and 2235 grams of sodium chloride are mixed with purified water in the tank, to make 95 liters of bicarbonate solution. After mixing with a stirrer, the concentrated solution is run into receptacles. The concentrate is prepared within 24 hours of its use.

A new formulation of Sodium thiosulfate containing bicarbonate mixture is proposed which would contain 1195 grams of Sodium thiosulfate, 6264 grams sodium bicarbonate and approximately 1990 grams of sodium chloride which can be mixed with purified water to a total volume of 95 liters. The amount of sodium chloride could be changed to achieve final sodium concentration in the desired range. The exact amount of sodium thiosulfate can be changed to deliver a prescribed amount of sodium thiosulfate in the final dialysate solution. This has to be mixed with acid solution (by mixing 17.7 Kilograms of GRANUFLOW® NATURALYTE® Dry Acid Concentrate or equivalent Acid concentrate mixed with purified water to a total volume of 62.5 liters) and purified water in a proportion of 1.83:1:34 to make a final dialysate containing sodium thiosulfate 62.5 mg/dl. This amount of sodium thiosulfate containing bicarbonate mixture, when used in conjunction with NATURALYTE® 9000 series Acid formulation or equivalent, will produce enough dialyzing fluid for approximately fifteen 4 hour time periods of dialysis at a maximum flow of 500 ml/min.

### New Sodium Thiosulfate Containing Dry Bicarbonate Mixture (Hereafter Called STS Bicarb Mix):

### Chemical Composition:

Sodium Bicarbonate 6290 grams
Sodium chloride 1990 grams
Sodium thiosulfate (STS) 1195 grams

### Directions for Use:

Mix 9715 grams of STS Bicarb mix powder with purified water that meets or exceeds the current AAMI/ANSI hemodialysis water quality standards to make the final volume 95 liters. Water temperature should be 24 degree Celsius. Mix the solution gently as the powder is being added. Vigorous mixing should be avoided. Ensure that powder is dissolved in solution. The container should be free of bacterial and chemical contamination according to current AAMI standards. The solution should be used within 24 hours of mixing.

This has to be used with NATURALYTE® 9000 series GRANUFLO® acid formulation or equivalent 17.7 kilograms mixed with purified water make a total volume of 32.5 liters and purified water in a proportion of 1.83:1:34 to make the final dialysate. Alternative formulations are also proposed for hemodialysis dialysate, and may be selected as Follows:

(1) Amount of STS can be changed depending upon the need and response to treatment in the bicarbonate powder mix without appreciably changing the other constituents.

(2) Bicarbonate STS mix can be prepared in smaller quantities. An example of the proposed bicarbonate STS can be obtained by adding 500 grams of sodium bicarbonate to 107.14 grams of sodium thiosulfate. Purified water meeting MMI/hemodialysis water quality requirements should be added to make total volume of 6 liters. This is mixed with NATURALYTE® or equivalent acid concentrate (with NaCl 214.8 g/L, KCl 5.22 g/L, CaCl₂ 4.86 g/L, MgCl₂ 1.67 g/L, CH₃CO₂H 6.31 g/L, glucose (C₆H₁₂O₆ 70 g/L) solution and purified water in a proportion of 1.225:1:32.775 by the dialysis machine to produce the final dialysate containing STS 62.5 mg/dL. This formulation would be more appropriate for an acute dialysis set up, home hemodialysis and in situations where the prescription has to be individualized. If more STS is desirable additional STS may be added to this bicarbonate STS mix. This additional amount of STS may be provided in sachets or vials containing 107.14 grams and any different quantities.

### New Peritoneal Dialysis Solution:

The composition of a standard 2000 ml peritoneal dialysis fluid is Dextrose 1.5 g/dL, 2.5 g/dL or 4.25 g/dL, sodium chloride 538 mg/dL, Sodium lactate 448 mg/dL, Calcium chloride 18.4 mg/dL, Magnesium chloride 5.1 mg/dL and water in sufficient quantity to make it 2000 ml. There may be small amount of hydrochloric acid or sodium hydroxide to maintain the pH between 5.0 and 6.0.

This would approximately give sodium 135 mEq/L, Magnesium 0.5 mEq/L, Calcium 2.5 mEq/L, Chloride 95 mEq/L, Lactate 40 mEq/L and no potassium. Solutions with higher magnesium and higher calcium are also available. Formulations containing icodextrin or amino acids instead of glucose are also available. There is another formulation available which has bicarbonate as the buffer instead of lactate.

New Proposed Formulations of Peritoneal Dialysate in this Invention Include:

1. Formulation containing dextrose as the osmotic agent in concentrations varying from 1.5 g/dL to 4.25 g/dL, sodium thiosulfate in varying concentrations from about 20 to about 300 mg/dL, other salts such as sodium chloride, sodium lactate, calcium chloride, magnesium chloride and sterile water in sufficient quantities to have sodium 132-135 mEq/L, Magnesium 0.25 to 0.75 mEq/L, Calcium 2.0 to 3.5 mEq/L, Chloride 95 to 106 mEq/L, Lactate 35 to 40 mEq/L and no potassium in the final dialysate.

2. Formulation containing icodextrin instead of dextrose as the osmotic agent in varying concentrations, sodium thiosulfate in varying concentrations from 15 to 300 mg/dL, other salts such as sodium chloride, sodium lactate, Calcium chloride, Magnesium chloride and sterile water in sufficient quantities to have sodium 132-135 mEq/L, Magnesium 0.25 to 0.75 mEq/L, Calcium 2.0 to 3.5 mEq/L, Chloride 95 to 106 mEq/L, Lactate 35 to 40 mEq/L and no potassium in the final dialysate.

3. Formulation containing amino acids instead of dextrose as the osmotic agent in varying concentrations, sodium thiosulfate in varying concentrations from about 20 to about 300 mg/dL, other salts such as sodium chloride, sodium lactate, calcium chloride, magnesium chloride and sterile water in sufficient quantities to have sodium 132-135 mEq/L, Magnesium 0.25 to 0.75 mEq/L, Calcium 2.0 to 3.5 mEq/L, Chloride 95 to 106 mEq/L, Lactate 35 to 40 mEq/L and no potassium in the final dialysate.

4. Formulation containing any other osmotic agent in varying concentrations, sodium thiosulfate in varying concentrations from 15 to 300 mg/dL, other salts such as sodium chloride, sodium lactate, Calcium chloride, Magnesium chloride and sterile water in sufficient quantities to have sodium 132-135 mEq/L, Magnesium 0.25 to 0.75 mEq/L, Calcium 2.0 to 3.5 mEq/L, Chloride 95 to 106 mEq/L, Lactate 35 to 40 mEq/L and no potassium in the final dialysate.

In general, conventional dialysates are defined as any formulation heretofore known, whether or not proprietary, including those that are recently patented. Many of these are specially formulated to satisfy the needs of a particular patient type. For example, U.S. Pat. No. 6,436,969 discloses compositions containing AGE inhibitors, U.S. Pat. No. 5,869,444 claims solutions contain an osmotically effective mixture of peptides, U.S. Pat. Nos. 6,306,836 and 6,380,163 disclose peritoneal dialysis solution utilizing amino acids to achieve osmotic balance, U.S. Pat. No. 5,968,966 provides replenishing levels of L-carnosine, U.S. Pat. No. teaches a formulation for improved ultrafiltration profiles having low salt content, and U.S. Pat. No. 6,551,990 incorporates osteopontin to prevent ectopic calcification. All of these may be used as a conventional base formulation for inclusion of sodium thiosulfate.

Dialysis is defined as the movement of solute and water through a semipermeable membrane (the dialyzer) which separates the patient's blood from a cleansing solution (the dialysate). Four transport processes may occur simultaneously during dialysis.

1. Diffusive transport is the movement of solutes across the membrane, and is dependent on the concentration gradient between plasma water and dialysate;
2. Convective transport is the bulk flow of solute through the dialyzer in the direction of hydrostatic pressure gradient;
3. Osmosis is the passage of solvent (water) across the membrane in the direction of the osmotic concentration gradient; and
4. Ultrafiltration is the movement of solute free water along the hydrostatic pressure gradient across the membrane.

The patient's plasma tends to equilibrate with the dialysate solution over time. The composition of the dialysate permits one to remove, balance or even infuse solutes from and into the patient. The electrochemical concentration gradient is the driving force that allows the passive diffusion and equilibration between the dialysate and the patient's blood compartment. The process of dialysis can be accomplished by using an artificial kidney (hemodialysis, hemofiltration or combination of these two processes) or patient's abdomen (peritoneal dialysis) or the mucous membrane of the gastrointestinal tract.

In an artificial kidney, a synthetic or semi-synthetic semipermeable membrane made of either cellulose acetate, cupraphane, polyacrilonitrile, polymethyl methacrylate, or polysulfone is used. A constant flow of blood on one side of the membrane and dialysate on the other allows removal of waste products. An artificial kidney can be used to perform hemodialysis, during which diffusion is the major mechanism for solute removal. On the other hand hemofiltration (also called hemodiafiltration and diafiltration) relies on ultrafiltration and convective transport rather than diffusion to move solutes across a high porosity semipermeable membrane. For the purposes of this application, the term hemodialysis is used to include all dialysis techniques (e.g. hemofiltration) that require an extracorporeal blood circuit and an artificial membrane.

On the other hand, peritoneal dialysis uses patient's peritoneal membrane to exchange solutes and fluid with the blood compartment. Therefore, peritoneal dialysis is the treatment of uremia by the application of kinetic transport of water-soluble metabolites by the force of diffusion and the transport of water by the force of osmosis across the peritoneum. The peritoneum is the largest serous membrane of the body (approximately 2 square meters in an adult). It lines the inside of the abdominal wall (parietal peritoneum) and the viscera (visceral peritoneum). The space between the parietal and visceral portions of the membrane is called the “peritoneal cavity”. Aqueous solutions infused into the cavity (dialysate) contact the blood vascular space through the capillary network in the peritoneal membrane. The solution infused into the peritoneal cavity tends to equilibrate with plasma water over time and it is removed at the end of one exchange after partial or complete equilibration. The composition of the dialysate permits to remove, balance or even infuse solutes from and into the patient. The electrochemical concentration gradient is the driving force that allows the passive diffusion and equilibration between the dialysate and blood compartment.

Gastrointestinal (hereafter referred to as ‘GI’) dialysis is an ancient dialysis modality and at this time it is not a standard therapy for ESRD in the United States. It has the benefit of being a very cheap and simple modality which can be done at patients home. Research is under progress to do GI dialysis using sorbents and probiotics. GI dialysis uses patient's mucous membrane of the gastrointestinal tract to exchange solutes and fluid with the blood compartment. Therefore, GI dialysis is the treatment of uremia by the application of kinetic transport of water-soluble metabolites by the force of diffusion and the transport of water by the force of osmosis across the mucous membrane. The gastrointestinal mucous membrane has a very large surface area and reasonable blood flow of 400 ml/minute in an adult. It lines the inside of the stomach, small intestine and large intestine. Aqueous solutions administered into the GI lumen (dialysate) contact the blood vascular space through the capillary network in the GI membrane. The solution administered into the GI tract tends to equilibrate with plasma water over time and it is excreted as bowel movement after partial or complete equilibration. In addition, the dialysate will flush away some of the toxins produced in the intestine which would otherwise have been absorbed. The composition of the dialysate permits to remove, balance or even infuse solutes from and into the patient. The electrochemical concentration gradient is the driving force that allows the passive diffusion and equilibration between the dialysate and blood compartment.

The dialysis solutions (hemodialysis or peritoneal dialysis or GI dialysis) of the present invention are characterized by an added compound, sodium thiosulfate. It has a molecular weight of 245, it is soluble in water and can easily transfer across the dialysis membranes.

Presently, hemodialysis machines utilize an automated proportioning system to mix salts in purified water in specific proportions to generate the final dialysate solution. The dialysate concentrates are usually supplied by the manufacturer either as a solution ready to use or as a premixed powder that is added to purified water in large reservoirs. The concentrates are pumped into a chamber in the dialysis machine where they are mixed with purified water to make the final dialysate solution.

Generally, the ionic composition of the final dialysate solution for hemodialysis is as follows: Na.sup.+, 132-145 mmol/L, K.sup.+0-4.0 mmol/L, Cl.sup.−99-112 mmol/L, Ca.sup.++1.0-3.0 mmol/L, Mg.sup.+2 0.25-0.75 mmol/L, Glucose 0-5.5 mmol/L. The correction of metabolic acidosis is one of the fundamental goals of dialysis. In dialysis practice, base transfer across the dialysis membrane is achieved by using acetate or bicarbonate containing dialysate. In “Bicarbonate dialysis” the dialysate contains 27-35 mmol/L of bicarbonate and 2.5-10 mmol/L of acetate. On the other hand, in “Acetate dialysis” the dialysate is devoid of bicarbonate and contains 31-45 mmol/L of acetate. Sodium thiosulfate is soluble in the dialysis solution.

The peritoneal dialysis fluid usually contains Na.sup.+132-135 mmol/L, K.sup.+0-3 mmol/L, Ca.sup.++1.25-1.75 mmol/L, Mg.sup.++0.25-0.75 mmol/L, Cl.sup.−95-107.5 mmol/L, acetate 35 mmol/L or lactate 35-40 mmol/L and glucose 1.5-4.25 gm/dL. Sodium thiosulfate is soluble in peritoneal dialysis solutions.

The composition of the GI dialysis fluid has to be determined, but it would somewhat equivalent to the peritoneal dialysis fluid. The big difference is that it does not have to be sterile. The osmotic agent could be different to avoid absorption of excessive amounts of fluid. Hence agents such as poly ethylene glycol, glycerol, and mannitol may be preferred over glucose or icodextrin. If absorption is desired amino acids and other nutrients may be added. STS content would be a same or little higher than that proposed for peritoneal dialysis solution since it going to be more intermittent.

In accordance with the present invention, sodium thiosulfate is either added directly to peritoneal or GI dialysis solutions, or to the concentrate for dialysate of hemodialysis. In case of hemodialysis, since the concentrates are diluted several fold in the machine by admixture with water; the compound has to be added at a proportionally higher concentration in the concentrate.

Currently, hemodialysis patients number about 400,000 in the United States and about one million worldwide. The majority of these patients are on hemodialysis, some are on peritoneal dialysis. GI dialysis is generally not practiced in any of the developed countries now. It has been documented that almost all of the adult hemodialysis population have increased coronary artery calcification score and ESRD has a very high cardiac mortality. Annual mortality rate of ESRD is much higher than many of the common cancers such as colon cancer, breast cancer or prostate cancer. Moreover patients with ESRD do not do as well as those without ESRD after coronary revascularization procedures. Hence dialysate sodium thiosulfate therapy is potentially useful for all dialysis patients.

Nephrogenic systemic fibrosis (hereafter called NSF, also called by different names such as Nephrogenic fibrosing dermopathy in the past) is a systemic disease characterized by progressive fibrosis of the skin, lungs, myocardium and striated muscles. It is observed in patients with renal insufficiency, most but not all of whom have been on dialysis. It is considered idiopathic, but there is an association between this and administration of intravenous Gadolinium, an agent used for magnetic resonance imaging. Because of this association, Food and Drug Administration (FDA) has issued a public health advisory regarding gadolinium containing contrast agents and a possible link to the development of NSF [Food and Drug Administration Public Health Advisory: Godolinium containing contrast agents for magnetic resonance imaging (MM): Omniscan, Opti MARK, Magnevist, ProHance, and MultiHance. (Updated 8 Jan. 2007) 2006. Available at the internet site of the FDA Accessed 12 Apr. 2007].Other regulatory bodies elsewhere in the world and the manufacturers of Gadodiamide have considered administration of gadolinium to be contraindicated in patients with a glomerular filtration of less than 30 ml/min/1.73 m². Insolubility and toxicity of Gadolinium can be potentially eliminated by forming chelates such as Gd-DTPA (Gadolinium-diethylene triaminepentaacetic acid) (Weinmann H J, et al., Roentgenol 1984; 142; 619-624). STS can form a chelate to Gadolinium to make it a soluble complex which can be excreted more easily. Even though Gadolinium dialyzable and can be removed almost completely in 4 successive dialysis, intradialytic STS will hasten the removal and reduce its tissue exposure.

The present invention provides pharmaceutical composition of a soluble compound that can be added to dialysis solutions to meet the therapeutic needs of dialysis patients in preventing or treating calcification of various tissues in the body thereby improving the high morbidity and mortality associated with ESRD. Since most of the tissue damage in uremia is mediated through calcification and oxidant injury, sodium thiosulfate with its chelating, antioxidant, reducing and antibrowning and cytoprotective properties would be an ideal agent to be administered with the dialysate. This method will also take away the additional cost of preparing intravenous medication and the cost of administration.

The following examples explain the preparation and utility of the present invention in certain typical situations.

### EXAMPLE 1

### Typical Representative Type Patient and Hereafter Examples

A 25 year old patient on hemodialysis presents with elevated coronary artery calcium (hereafter called CAC) score detected during an Electron Beam Computerized Tomogram (hereafter called EBCT) done as a part of an executive physical examination. Patient has no cardiac symptoms and has no history of known coronary disease. Knowing that a 20 years old dialysis patient carries annual mortality rate equivalent to that of a 70 years old non-dialysis patient, it is only prudent to modify his risk factors for clinically significant coronary artery occlusion.

According to the present invention this patient will be able to get a treatment with intradialytic sodium thiosulfate which specifically would remove the calcium deposits in the coronary arteries and reduce the risk of future coronary events. He could initially be started on the formulation delivering STS 62.5 mg/dL in the final dialysate. CAC score would be repeated in 12 months. If CAC score improves the same dialysate may be continued. If the CAC score does not improve significantly, the STS could be increased to deliver approximately 125 mg/dL.

Sodium thiosulfate is a whitish translucent crystalline compound that is known to be soluble in water. Hemodialysis with STS containing dialysate does result in transfer of STS to the blood compartment. STS in the blood binds to the calcium phosphate and other calcium salts to form calcium thiosulfate. Calcium thiosulfate, being many folds more water soluble than calcium phosphate, diffuses back to the dialysate compartment and is excreted.

Recommended dosage: The amount of sodium thiosulfate will be calculated to achieve a final concentration of 62.5 mg of STS per deciliter of final dialysate obtained after mixing the purified water, bicarbonate mix solution and the acid mix solution. This concentration of STS could be adjusted depending upon the result achieved and desired.

Monitoring: Applicant recommends that the coronary artery calcium (hereafter called CAC) score be measured periodically (approximately every 12 months till the CAC returns to normal range and then every 2 year) and if the CAC does not come down at least by 20% in 12 months the concentration of STS may be increased. Since CAC score is elevated in large majority of the adult hemodialysis patients it would also be reasonable to administer STS in the dialysate without monitoring the CAC score.

### EXAMPLE 2

A 50 year old female on hemodialysis for 2 years comes asking her prognosis. She is unable to get kidney transplantation for medical or religious reasons. After knowing her prognosis, she requests that we do everything to improve her odds of doing well on hemodialysis. Her insurance company does not pay for EBCT scan to measure CAC score and she cannot afford to pay for it herself. Knowing that she carries a high risk of cardiovascular events in future, we would try to modify the conventional risk factors for cardiovascular events. Of note is that even though statins are still prescribed in such situation if cholesterol level is high, studies have not shown any mortality benefit of statins in hemodialysis patients. In addition, it would be reasonable to put her on STS containing dialysate knowing that majority of the adult hemodialysis patients have a high CAC score.

This practice could be applied to all the adult patients on hemodialysis. Hence the formulation containing STS can be used for all the patients in hemodialysis in outpatient dialysis units.

### EXAMPLE 3

A 50 year old male on hemodialysis presents with recent onset of shortness of breath on minimal exertion. Detailed evaluation revealed that she has pulmonary hypertension and restrictive pattern on pulmonary function tests. Patient has no other known risk factors for pulmonary hypertension and restrictive lung disease. Conventional treatment to improve the pulmonary hypertension is not very effective in this situation. In addition, she could be offered a definitive treatment which has the potential for removing the calcium deposition from the pulmonary vasculature and lung parenchyma. This could improve lung compliance, gas exchange and right ventricular/pulmonary artery pressure.

STS may also help improve this situation by its antioxidant and vasodilatory properties.

Dosage and monitoring: Initially these patients could be placed on dialysate delivering 62.5 mg/dL of STS. In these patients echocardiogram with Doppler pressure measurement pulmonary artery pressures and pulmonary function tests will have to be monitored approximately every 12 months. Other ways of monitoring could be added depending upon the concurrent organ damage. Depending upon the response in about 12 months decision could be made to continue same dose of STS or increase the amount of STS in the dialysate.

### EXAMPLE 4

An 80 year old patient on hemodialysis is found to have calcified aorta and mesenteric arteries during a routine abdominal x-ray done for evaluation of abdominal pain. Knowing that calcification of the arteries often precedes ischemic events, such patients could be offered she could be offered a definitive treatment which has the potential for removing the calcium deposition from the vasculature.

Dosage and monitoring: Intradialytic STS could be used in any concentration of physicians' choice with minimal monitoring similar to example 1.

### EXAMPLE 5

A 35 year old man on home hemodialysis requests for maximal effort to improve his prognosis.

In addition to the routine care this patient could be offered dialysate with STS to remove the existing vascular and ectopic calcification and prevent further calcification.

This patient could be monitored by yearly CAC score, but one could chose to have no monitoring by imaging studies besides the routine care.

### EXAMPLE 6

A 40 year old woman is undergoing training for peritoneal dialysis at home. Routine care would involve using conventional dextrose containing dialysate. Instead this patient could be offered STS containing dialysate. In addition to the potential systemic benefits of STS, this may also reduce the dialysis related injury to the peritoneal membrane making it last longer. This could potentially increase the technique survival of peritoneal dialysis.

### EXAMPLE 7

15 year old female on peritoneal dialysis presents with elevated CAC score: In addition to the conventional bone and mineral management, a specific therapy to improve the vascular calcification in the form of STS containing dialysate could be offered. Yearly monitoring of CAC score and adjustment of STS concentration in the dialysate depending upon the response is recommended. In addition yearly peritoneal equilibration test or some other peritoneal function test is recommended.

### EXAMPLE 8

40 year old living abroad on gastrointestinal dialysis:

As such GI dialysis is an inferior therapy than peritoneal of hemodialysis. STS containing dialysate in this patient could benefit such patient by its systemic benefits as well as local effects on the GI mucous membrane. This has the potential of helping save lives in situations where hemo- and peritoneal dialysis are not feasible.

The optional monitoring would include yearly CAC score and if the CAC does not come down at least by 20% in 12 months the concentration of STS may be increased. Since most of these patients have risk for coronary calcification, it would be reasonable to continue STS for the rest of their lives. However, stopping STS once the CAC returns to normal range and restarting it if CAC score goes up would be guardedly reasonable.

### REFERENCES

1. K/DOQI clinical practice guidelines for chronic kidney disease: evaluation, classification, and stratification. Am J Kidney Dis. February; 39(2 Suppl 1):S1-266.
2. www.usrds.org
3. Goodman W G, Goldin J, Kuizon B D et al. Coronary artery calcification in young adults with end stage renal disease who are undergoing dialysis. N Eng J Med 2000; 342: 1478-1483.
4. Raggi P, Boulgy A, Chason-Tober S, Amin N, Dillon M, Burke S K, Chertow G M. J Am Coll Cardiol, 2002; 39:695-701
5. Vliegenthart R, Hollander M, Breteler M M B, van der Kuip D A M, Hofman A, Oudkerk M, Witteman J C M. Stroke is associated with Coronary Artery Calcification detected by Electron beam CT. The Rotterdam Coronary Calcification Study. Stroke. 2002; 33:462.
6. Vlierenthart R, Oudkerk M, Song B, van der Kuip D A M, Hofman A, Witteman J C M. Coronary Calcification detected by electron beam computed tomography and myocardial infarction. The Rotterdam Coronary Calcification Study. European Heart Journal 2002 23(20): 1596-1603
7. Moe S M, Chen N X: Calciphylaxis and vascular calcification: A continuum of extra-skeletal osteogenesis. Pediatr Nephro/18:969-975, 2003
8. O'Neill W C. Vascular calcification: Not so crystal clear. Kidney International (2007) 71, 282-283.
9. Verbercknoes S C, Persy V, behets G J, et al Kidney International (2007) 71, 298-303.
10. Yatzidis H: Successful sodium thiosulphate treatment for recurrent calcium urolithiasis. Clin Nephrol 23:63-67, 1985.
11. Agroyannis B J, Koutsikos D K, Tzanatos H A, Konstadinidou I K: Sodium thiosulphate in the treatment of renal tubular acidosis I with nephrocalcinosis. Scand J Urol Nephrol 128:107-108, 1994.
12. Brucculeri M, Cheigh J, Bauer G, Serur D: Long-term intravenous sodium thiosulfate in the treatment of a patient with calciphylaxis. Semin Dial/8 :431-434, 2005.
13. Braverman B, Ivankovich A D, Shah G: Thiosulfate pharmacokinetics in normal and anuric dogs. Proc Soc Exp Biol Med 170 :273-280, 1982.
14. Cardozo R H, Edelman I S: The volume of distribution of sodium thiosulfate as a measure of the extracellular fluid space. J Clin Invest 31 :280-290, 1952.
15. Food and Drug Administration Public Health Advisory: Godolinium containing contrast agents for magnetic resonance imaging (MRI): Omniscan, Opti MARK, Magnevist, ProHance, and MultiHance. (Updated 8 Jan. 2007) 2006. Available at http://www.fda.gov/cder/drug/advisory/gadoliniumagents.htm. Accessed 12 Apr. 2007].
16. Weinmann H J, et al., Roentgenol 1984; 142; 619-624.
17 .neuropathy. Med Sci Monit 2004, 10(12): RA291-307
18. Kyriakopoulos G, Kontogianni K: Sodium thiosulfate treatment of tumoral calcinosis in patients with end-stage renal disease. Ren Fail 12 :213-219, 1990.
19. Guerra G, Shah R C, Ross E A: Rapid resolution of calciphylaxis with intravenous sodium thiosulfate and continuous venovenous haemofiltration using low calcium replacement fluid: Case report. Nephrol Dial Transplant 20 :1260-1262, 2005.

1. A method of treating dialysis dependent patients suffering from end-stage renal disease in which said patient has or is_susceptible to complications from calcification processes in the body comprising:
mixing a hemodialysate solution from a dry powder mixture comprising sodium thiosulfate so that said hemodialysate solution comprises about 20 mg/dl to about 130 mg/dl sodium thiosulfate,
performing hemodialysis on said dialysis dependent patient with said hemodialysate solution, and assessing said dialysis patent for reduction of calcification.

2. The dialysis method of claim 1 wherein said calcification processes include vascular calcification characterized by coronary ischemic events, peripheral vascular occlusive disease, mesenteric ischemia, restrictive lung disease or pulmonary hypertension, skin ulcers, decubitous ulcers, and other organ vascular insufficiency; myocardial, endocardial and pericardial calcification, calcification of the heart valves; muscular calcification; and calcification of perineurium, endoneurium, the vasa nervorum, distal pre-capillary arterioles, and soft tissue.

3. The method of claim 1, wherein said assessing said dialysis dependent patient for reduction of calcification comprises determining the coronary artery calcium score of said patient.

4. The method of claim 3, wherein said coronary artery calcium score of said patient is improved as compared to a coronary artery calcium score of said patient prior to said hemodialysis.

5. A method of treating a dialysis dependent patient suffering from end-stage renal disease in which said patient has or is susceptible to complications from calcification processes in the body comprising:
providing a hemodialysate solution comprising about 20 mg/dl to about 130 mg/dl sodium thiosulfate,
performing hemodialysis on said dialysis dependent patient with said hemodialysate solution and
assessing said dialysis dependent patient for reduction of calcification.

6. The method of claim 5, wherein said assessing said dialysis dependent patient for reduction of calcification comprises determining the coronary artery calcium score of said patient.

7. The method of claim 6, wherein said coronary artery calcium score of said patient is improved as compared to a coronary artery calcium score of said patient prior to said hemodialysis.
